(19) United States
(12) Patent Application Publication
(10) Pub. No.: US 2005/0227086 A1
(43) Pub. Date: Oct. 13, 2005

(54) Water vapor permeable, water impermeable barrier sheet member

(76) Inventor: Donald J. Murphy, Valdosta, GA (US)

Correspondence Address: ROGERS TOWERS, P.A., 1301 RIVERPLACE BOULEVARD, SUITE 1500, JACKSONVILLE, FL, 32207, US

(21) Appl. No.: 10/819,733

(22) Filed: Apr. 7, 2004

(51) Int. Cl. A61F 13/15

(52) U.S. Cl. 428/423.1

(57) ABSTRACT

A composite or multi-layer sheet material, suitable for use as a housewrap material, and the manufacturing process therefore, comprising an extruded pressure sensitive adhesive disposed between a polypropylene fabric layer, woven or non-woven, and a polyurethane layer. Preferably a scrim reinforcing layer is disposed between the polypropylene layer and the polyurethane layer for added strength and tear resistance. A non-slip layer may be added to the exterior side of the polypropylene layer for certain applications, such as roof underlayment. The material is an absolute barrier to liquid water transmission that has moderate permeability to water vapor of greater than 35 grams/sq. meter/24 hrs, and most preferably between approximately 105 to 210 grams/sq. meter/24 hrs. The sheet material may be manufactured by extruding the pressure sensitive adhesive between the polypropylene and polyurethane layers, but is preferably produced by co-extruding the pressure sensitive adhesive and polyurethane onto the polypropylene layer, with the scrim reinforcing layer disposed therebetween.

### BACKGROUND OF THE INVENTION

The present invention relates generally to a polymer and fabric composite forming a water vapor permeable, water impermeable barrier sheet member, and more specifically to a continuous co-extrusion coating applied to a woven or nonwoven synthetic fabric backing, which is particularly useful as a barrier to air and moisture infiltration of residential and commercial building structures.

Moisture barriers are generally required by building code regulations as a secondary means of preventing moisture intrusion into the structure upon failure of the primary exterior cladding as a result of cracks, unsealed joins, weathering and ground settling. The resulting damage from entry of moisture into the wall cavity may be considerable, frequently resulting in wood rot, mildew, moisture damage in insulation and termite infestation. There are three types of materials that are generally accepted as moisture barriers: asphalt saturated roofing felt, asphalt saturated building paper and more recently introduced since the 1980's, a number of products consisting of woven, nonwoven or spun bonded polymer based materials commonly referred to as housewraps. Asphalt saturated roofing felt has been widely used over a number of years, however, its usefulness is limited by the narrow width of the material resulting in additional labor costs, the necessity to lap the material resulting in voids in the barrier membrane application and its limited water resistance. Asphalt saturated building paper has similar limitations, is considerably thinner that roofing felt yet it is a more homogeneous composition that approximates the water resistance values of roofing felt. Polymer-based housewrap materials are known to be produced in three variations: extruded polyethylene film with micro perforations, polymer coated synthetic fabrics with micro perforations, and various composite products consisting of micro porous films. An example of this type of housewrap is a product from the E.I. DuPont Company based on a spun bonded micro porous film structure and marketed under the brand name Tyvek HomeWrap, Tyvek Commercial Wrap and Tyvek Stucco Wrap.

It is necessary that all moisture barriers and housewrap materials be breathable with a minimum permeability to water vapor of 35 grams/sq. meter/24 hrs. (ASTM E-96 Procedure A or B). Some extruded films and coated woven synthetic fabrics attain their permeability by micro perforations which, as applied in a vertical plain, resist moisture penetration with the exception of the most severe conditions, nonetheless they cannot be considered absolute barriers to water penetration. Most polymer-based films, specifically polyethylene, are inherently water resistant, however they are likewise barriers to water vapor transmission with the exception of the thinnest gauges, which would not be practical for use given the code stipulated strength requirements for moisture barrier materials. Micro porous polyethylene films attain a breathable characteristic by a dual process whereby the film is first extruded with a filler such as calcium carbonate and then stretched monaurally, thereby orientating the molecules of the film in the machine direction. This process allows for gaps or interruptions in the molecular structure of the film, allowing water vapor to diffuse by means of the differential in vapor pressure from the higher pressure entry side of the film to the lower pressure exit side of the film. A spun bonded structure is likewise classified as being micro porous, achieving micro porosity in the initial manufacturing process.

Water will generally not penetrate micro porous film due to the fact that the microscopic openings are too small (approximately 1 mil) to overcome the surface tension of water that would otherwise allow water to penetrate. However, a significant problem has been noted in many films of this nature. In building structure applications there are several instances where the material comes in contact with surfactants and wood extractives that effectively destroy the water resistant properties of the micro porous film surface, thereby allowing undesirable water penetration. Surfactants are used in various construction materials such as stucco, mortar, treated lumber, wood preservatives, paints, soaps and detergents, as would be normally used for power washing exteriors of homes and structures. Brick exteriors are inherently porous and present a particular problem since cement and mortar mixes contain surfactant chemicals to extend open time, especially under low temperature conditions. The surfactants found in these materials will reduce the surface tension of water, allowing penetration through the micro porous openings in a micro porous film or spun bonded polyolefin. Consequently, this becomes a serious problem since the reverse side of wood siding and structural wood framing lumber as well is often treated with these wood preservatives. Further, extractives from red wood and cedar siding in contact with housewrap materials based on a micro porous film composite have shown loss of water barrier properties as evidenced by tests conducted by the Forest Products Department at the University of Massachusetts at Amherst.

Water resistance is not the only important property that is necessary for a high performance level of a housewrap material. Frequently, delays in the completion of a home or structure make it necessary for the housewrap to be the only weather barrier for several months. Therefore, it is necessary that a housewrap material to be able to withstand heavy rain, high winds and the effect of UV degradation over a period of several months. Safety is another very important factor in the properties of a material, specifically, a non-slippery surface that would minimize any potential for ladder accidents is most desirable.

The material of the invention is also suitable for a second type of application in the building industry, that of underlayment for exterior roofing materials. For almost a hundred years, the building industry has utilized 15 and 30 pound roofing felt as an underlayment for exterior roofing materials. Roofing felt is heavy, weighing 15 and 30 pounds per 100 square feet of materials, tears easily, and becomes stiff, brittle and hard to handle low temperatures. Roofing felt will also oxidize over a period of time losing its strength and the ability to withhold water. The oxidation occurs by the drying out or loss of the volatile oils in the asphalt due to heat and years of exposure. A very significant factor in the drawback of use of roofing felt however is the difficulty in handling and applying the material.

Recently several companies have introduced synthetic or polymer based alternative materials for use as roofing underlayments. In prominent use is a non-woven polypropylene fabric that is extrusion coated on both sides with a polypropylene coating containing an additive for slip resistance. Another product is based on a polypropylene coating of a woven polypropylene fabric. These materials are offered in similar widths as roofing felt but offer a significant advantage of weighing approximately 1/10^{th} of the weight of 30 pound roofing felt. Priced competitively with roofing felt, these products have been well received primarily because of their lighter weight and ease of application.

It is significant to note that ASTM D-4869, which is the test specification for the properties of roofing felt, states a minimum water vapor permeability of 35 grams per square meter per 24 hours. The purpose of this requirement is to prevent condensation problems resulting from the escape of warmer moist air from the interior of the structure with condensation occurring on the cold side of the roof resulting in potential wood rot and water leakage. This is primarily a problem in colder climates when an attic area is provided with insufficient venting, resulting in a frost line building up on the surface of the roof sheathing at night and then melting during the daylight hours on exposure to sunlight. This type of cycling during winter months can result in serious structural damage, very similar to the occurrence of ice standing along the eaves of a roof. It is therefore very important that a roofing underlayment material possess the characteristic of moderate permeability to water vapor to allow for the escape of otherwise entrapped warmer moist air from the interior and thereby prevent occurrence of condensation. A well insulated underside of roof trusses, together with the installation of a good vapor barrier, will minimize any problems resulting from the installation of a barrier-like underlayment under these extreme weather conditions, however these measure are more the exception than the rule.

What is proposed with this invention is an exceptionally high strength material suitable for housewrap or underlayment applications with moderate, controlled water vapor permeability, impermeable to water penetration, unaffected by contact with surfactants or chemicals, a low coefficient of friction for greater worker safety in applying the material, and economical in cost.

### BRIEF DESCRIPTION OF THE DRAWINGS

FIG. 1 is a cross-sectional view of a basic embodiment of the composite or multi-layer sheet material.

FIG. 2 is a cross-sectional view of a preferred embodiment of the invention incorporating a scrim reinforcing layer.

FIG. 3 is a cross-sectional view of another preferred embodiment of the invention incorporating a FIG. 4 is a diagram showing a first embodiment of a system illustrating the process for manufacturing the invention.

FIG. 5 is a diagram showing a second embodiment of a system illustrating the process for manufacturing the invention.

FIG. 6 is a diagram showing a third embodiment of a system illustrating the process for manufacturing the invention.

### SUMMARY OF THE INVENTION

The present invention provides for a woven or nonwoven polyolefin base fabric with a polymer coating possessing distinct features and improvements over present art for use as a moisture-air filtration barrier membrane covering for residential and commercial building structures. The material is an absolute barrier to liquid water transmission that has moderate permeability to water vapor of greater than 35 grams/sq. meter/24 hrs, and most preferably between approximately 105 to 210 grams/sq. meter/24 hrs (ASTM E-96, Procedures A or B).

The invention is a composite or multi-layer sheet material, suitable for use as a housewrap material, and the manufacturing process therefore, comprising an extruded pressure sensitive adhesive disposed between a polypropylene fabric layer, woven or non-woven, and a polyurethane layer. Preferably a scrim reinforcing layer is disposed between the polypropylene layer and the polyurethane layer for added strength and tear resistance. A non-slip layer may be added to the exterior side of the polypropylene layer for certain applications, such as roof underlayment.

The sheet material may be manufactured by extruding the pressure sensitive adhesive between the polypropylene and polyurethane layers, but is preferably produced by co-extruding the pressure sensitive adhesive and polyurethane onto the polypropylene layer, with the scrim reinforcing layer disposed therebetween.

### DETAILED DESCRIPTION OF THE INVENTION

With reference to the drawings, the invention will now be described in detail with regard for the best mode and the preferred embodiments.

Polypropylene is a very versatile and cost efficient polymer that is widely used in the production of both slit ribbon yarn woven fabric as well as spun bonded nonwoven fabrics for both construction application uses such as moisture barriers (i.e. housewrap), vapor barriers, concrete curing covers as well as numerous industrial packaging applications. Polypropylene has been found to be the polymer of choice for properties of stiffness, dimensional stability at high and low temperatures, tensile strength and impact resistance. In addition to construction and industrial applications, polypropylene nonwoven fabrics are widely used in consumer and medical complications such as diapers, dryer softener, wipes, surgical drapes and medical apparel. In many of these applications, breathability is an essential factor, which requires the lamination or coating of a breathable surface coating or lamination to achieve a permeability level sufficient for personal comfort and avoidance of accumulated perspiration. In construction applications, as well, it is necessary to utilize membranes that are breathable to prevent accumulation of water vapor within the exterior wall cavity that will result in condensation and structural damage due to moisture build-up, mildew and wood rot.

Urethane elastomers are particularly well suited as a fabric coating because of the excellent properties of water impermeability and moisture vapor permeability, however, it has been found that satisfactory bonding or adhesion is only achievable with a polyester based substrate. Polyurethane is highly incompatible for bonding (i.e. adhesion) to nonwoven and woven polypropylene fabrics. Extensive testing and research has been conducted in an attempt to achieve an acceptable bond level with a polypropylene fabric which offers otherwise excellent strength properties and is available at a considerably lower cost than polyester based fabrics. Tie layer materials that might be considered for coextrusion with polyurethane include ethylene methyl acrylate (EMA), methylpentene (TPX) and copolyetherester (TPE-E). All of the these film resins are vapor permeable but have properties that are not adequate for the contended purpose as a weather resistant, vapor permeable coating for a woven or nonwoven polypropylene fabric. EMA, while having adequate bonding to polypropylene, has a moisture vapor transmission rate (MVTR) lower than the minimum required value of 35 grams/sq. meter/24 hrs. Likewise, TPX has a minimum satisfactory adhesion to polypropylene fabric but does not meet the required moisture vapor transfer rate under conditions of method A & B of ASTM E-96, which calls for a temperature of 73° F. at 50% relative humidity. It is only at condition E at a temperature of 100° F., 90% RH that TPX attains or reaches a MVTR value above 35 grams/sq. meter/24 hrs. TPE-E resin has good permeability values but less than satisfactory adhesion to polypropylene fabrics, and with the exception of black opaque film coatings, TPE-E has poor UV resistance, a property essential for many end use applications such as housewrap materials.

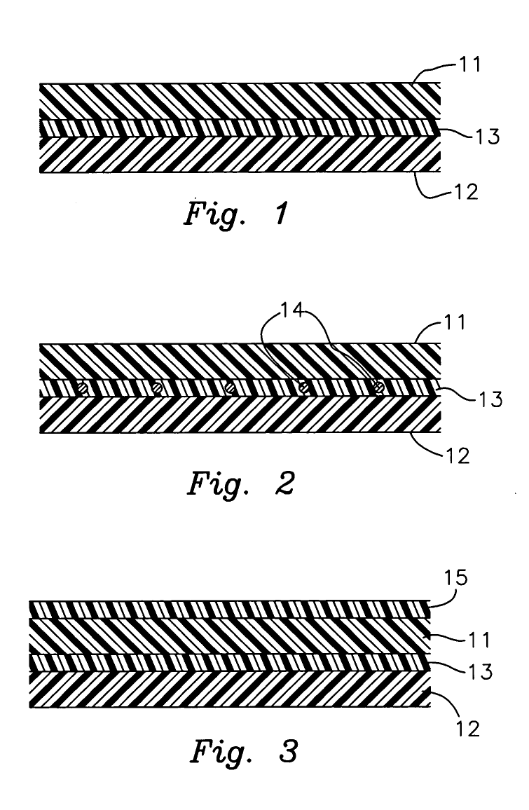

The invention, as illustrated in FIG. 1, is a composite or multi-layer sheet material comprising a polypropylene layer 11, a polyurethane layer 12, and a thermoplastic pressure sensitive adhesive (PSA) layer 13 disposed therebetween, where the PSA is extrudable or co-extrudable with the polyurethane. A suitable thermoplastic polyurethane, for example, is produced by the B.F. Goodrich Co. under the brand name Estane 58315. Other polyurethanes with similar properties could also be utilized.

The PSA acts as a tie layer, having bonding compatibility with both the polypropylene woven or nonwoven fabric layer and a polyurethane film layer, preferably in a co-extruded coating. While the PSA's primary component is a styrene block copolymer, which is basically a barrier material, it has been found that a minimal co-extrusion layer of approximately 0.00015 to 0.00035 inches in thickness in combination with a polyurethane layer with a thickness in the range of approximately 0.00055 to 0.000060 inches will have a MVTR value well in excess of 35 grams/sq. meter/24 hrs when tested in accordance with ASTM E-96, Procedure B. A suitable PSA, for example, is produced by the H.B. Fuller Co. under the brand Propel HL-2688. Other PSA's with similar processing and adhesion characteristics could also be utilized to bond the polypropylene layer 11 to the polyurethane layer 12. This PSA has high temperature resistance, including a SAFT (Shear Adhesion Failure Temperature) of 340° C., as well as having good flexibility at sub-zero temperatures.

The coating weight and gauge ratio of the polyurethane polymer and PSA co-extrudant has demonstrated excellent production efficiency and physical property test values when co-extruded at a ratio of approximately 4 to 1 polyurethane to PSA, with a total coating weight in the range of approximately 0.75 to 1 mil on the polypropylene layer. The multi-layer or composite sheet material has a MVTR value greater than 35 grams/sq. meter/24 hrs, and most preferably between approximately 105 to 210 grams/sq. meter/24 hrs.

In an improved and preferred embodiment, as shown in FIG. 2, a scrim reinforcing layer 14 of common composition is disposed between the polypropylene layer 11 and the polyurethane layer 12 in order to increase resistance to tear.

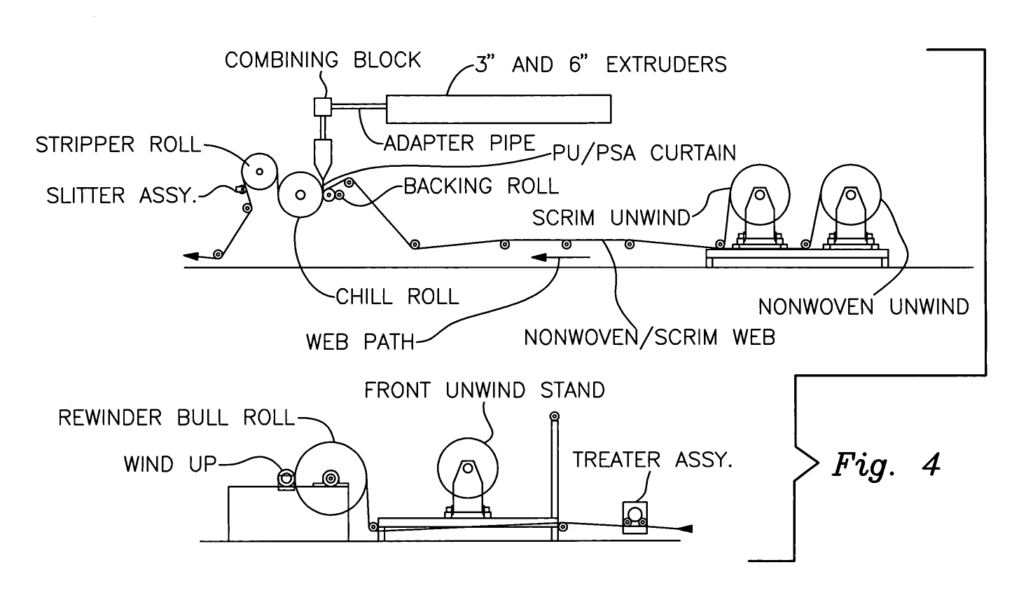

In an alternative embodiment, as shown in FIG. 3, the sheet material is provided with a non-slip or friction surface layer 15, such as for example ethylenemethylacrylate, making it especially suitable as underlayment for roofing applications. The testing of the material has demonstrated excellent properties of strength including tensile, tear, burst and puncture, resistance to failure at both high and low temperatures, optimum permeability of water vapor and impermeability to liquid water transmission. Further, the high coefficient of friction allows for a firm grip of the roll of material allowing for the necessary degree of tension to assure a tight, wrinkle-free application eliminating billowing, wind stress and eventual tearing as would otherwise occur with a slick, non-reinforced lower strength material. Further, the high friction surface allows for greater pressure of worker safety during application and avoidance of potential ladder accidents.

This material has a weight of approximately 30 pounds per thousand square feet of material. This is much lighter than when compared to a 300 pound basis weight for 30 pound roofing felt.

The water impermeable, water vapor permeable multi-layer sheet material as described above may be produced by several alternative processes, as illustrated in FIGS. 4 through 6.

A first process, illustrated by the equipment diagram of FIG. 4, comprises the steps of coating the woven or non-woven polypropylene fabric by co-extrusion of the polyurethane and PSA layers, applying the PSA co-extrudant against the scrim layer disposed on the polypropylene layer, such that the top layer of polyurethane provides water resistance, moderate permeability and high step resistance for the polypropylene layer.

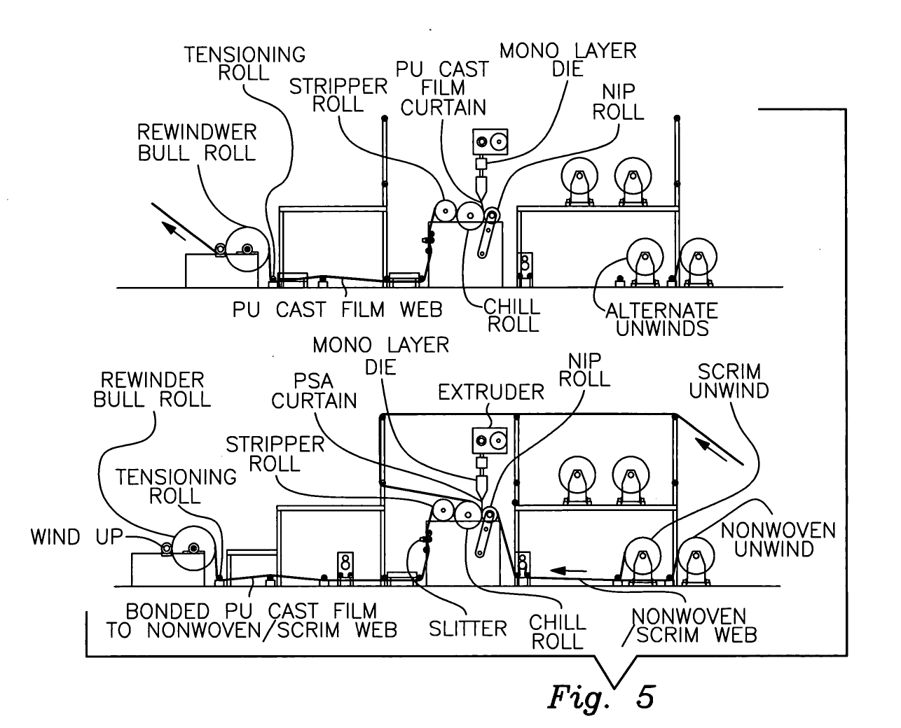

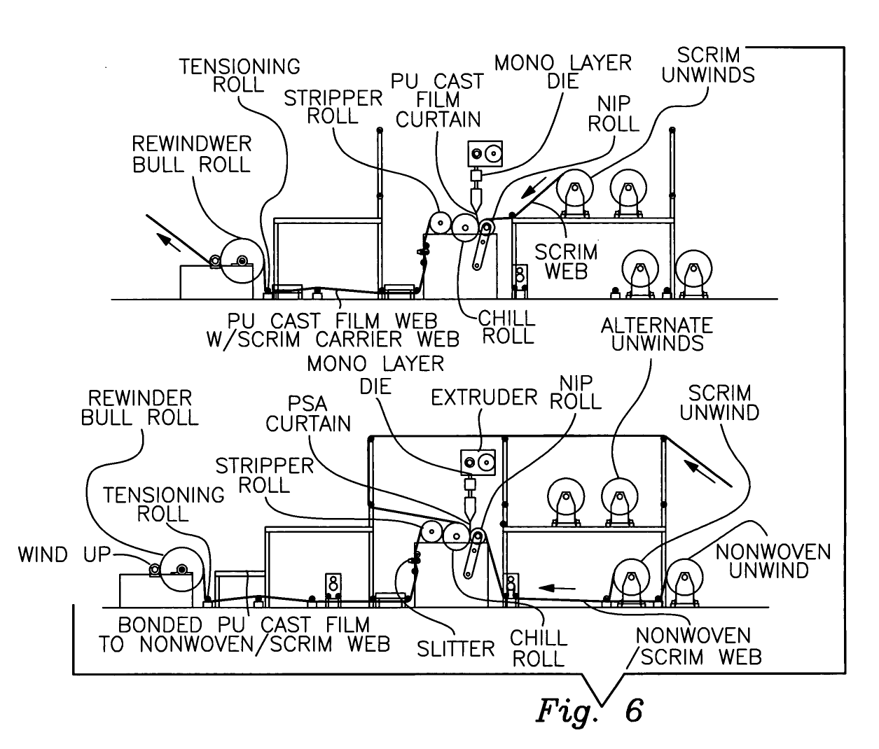

A second process, illustrated by the equipment diagram of FIG. 5, comprises coating the woven or nonwoven polypropylene fabric by a mono extrusion process in tandem. A film of polyurethane is first produced and then conveyed over idler rolls to a second coating station. At this station there are two unwind rolls, the first containing the woven or nonwoven polypropylene fabric and the second a woven or nonwoven scrim fabric. The scrim fabric is laid over the polypropylene fabric. The combined scrim and polypropylene components are then coated at the second coating station with a thin (preferably approximately ¼ mil) coating of the pressure sensitive adhesive. In the third step of the process, the cast polyurethane film from the first station is then combined by lamination with the scrim/fabric and wound into a finished roll.

The third process, illustrated by the equipment diagram of FIG. 6, is similar to the second process in that two extruders in tandem are used, with the exception that the cast polyurethane film is combined in route to the second coating station with the open scrim fabric, with the polyurethane film being in the upper plane. At the second coating station, the woven or non-woven fabric is coated directly with the PSA and combined with the cast film/scrim in the lamination process.

The following is a description of the equipment and production process for the co-extrusion coating of polypropylene woven and nonwoven fabrics with a thermoplastic polyurethane film resin and an extrudable pressure sensitive adhesive.

The die design which has been found to be most suitable for the flow characteristics of thermoplastic polyurethane (TPU) and an extrudable pressure sensitive adhesive (PSA) resins, is a coat hanger flow pattern with a tear drop cross section.

A heated transition line carries a melted resin from each extruder head to a heated combining block which proportionally layers two materials. The combining block, in-turn, is connected to a manifold die. The transition line, combining block and die are heated at the same temperatures by means of electrical resistance cartridge heaters. Since polyurethane is highly hygroscopic, it is necessary to dry the material to avoid gels and voids in the film coating and to achieve the best running conditions and higher film properties. A resin dryer is set a temperature of 550° F. for the desiccant and 150° F. for the resin chamber. The polyurethane will have a shelf life in the dryer no more than 12 hours and no longer than 15 to 20 minutes in the resin hopper. Exceeding the time factors will degrade the material, turning the resin yellow and causing voids and gel in the film curtain. Polyurethane resin must also maintain a constant control temperature in order to achieve the best quality film. The recommended temperatures are as follows:

| | | |
|---|---|---|
| Barrel Zone #1- | Barrel Zone #2- | Barrel Zone #3- |
| 330° F. | 350° F. | 370° F. |
| Barrel Zone #4- | Barrel Zone #5- | Barrel Zone #6- |
| 380° F. | 380° F. | 380° F. |
| Screen Changer- | Transition Zone- | |
| 380° F. | 380° F. | |

Due to the extreme differences in the melt flow and melt index between both the TPU and PSA resins, it has been found that the polyurethane coating will dominate the PSA curtain, taking precedence over the die and starving the PSA layer of the film curtain from coming out completely to the edge of the die. To compensate for this problem we have found that the flow inserts component in the combining block be modified by milling to a larger opening, that's increasing the flow of the PSA out to both edges of the die.

The die lips are V-contoured to minimized the air gap between the die and the roll nip whenever necessary.

The uncoated polypropylene and scrim substrate is led over the pressure roll where it meets the hot melt cascade flowing downward from the die. The pressure roll activated by a pair of pneumatic or hydraulically loaded air cylinders, forces the substrate and the hot melt together in the roll nip.

Adhesion and appearance can be controlled to a degree by using the rubber pressure rolls of varying hardnesses.

The pressure roll with an 85 durometer hardness is cooled both by internal circulating high velocity water at 76° F. and by playing a water-cooled aluminum roll against the trailing edge of the pressure roll as a heat sink.

The chill roll freezes the molten plastic to the substrate almost instantaneously; therefore, it must have a very sophisticated water cooling system. Its controlled speed determines film thickness and overall coating efficiency, and its surface finish determines the texture of the coating.

Commercial controllable line speeds can range from 30 ft./min. to 1200 ft./min.

Sophisticated tensioning, positioning and aligning devices are normally installed between the unwind and wind-up stations to ensure flat, smooth-edged rolls at high production speeds. Flying splice equipment is necessary to have long, continuous production runs at cost efficient line speeds.

Resin must be dried in the dryer for approximately 2-3 hours before running production. Fill the dryer with at least 2000 lbs. of resin to insure the suction hose valves are shut off when the hoses are not in use. Over-drying the resin will degrade it, so do not exceed 12 hours drying time in the resin chamber. All the resin in the drying chamber should be used up within this time (12 hrs.). Change out the chill roll (must have the matte chill roll in to run this product). A matte finish roll produces a non-reflective dull surface finish, which is more desirable since a bright, shiny finish on a white surface under sunlight conditions is an eye strain problem to construction workers applying housewrap material. Have the machine super clean—idler rolls, bearing chill roll, etc. Replace the Teflon tape, if it is worn, have the shear cuts, and the score cuts ready to trim to 108″ finished width.

Change the screen packs, put in a 20-80-20 in each pack (3.5 &6.0), drool polypropylene at 400° F. in both screws (3.5 & 6.0). When you are ready to run production, run the TPU resin in the 6.0 screw and the PSA in the 3.5 screw; insure that your PSA is up against the fabric (right hand valve open, left hand valve closed) and the TPU side is up against the chill roll.

The TPU resin has only a 15 minute shelf life in the 6″ hopper—if it sits any longer than 15 minutes it my have to be dumped out and redried, so it is of the utmost importance that everything is in its place and ready to run trouble free. The PSA is somewhat sticky and can cause problem feeding at the hopper throat. To eliminate this problem remove the hopper magnet and do not allow the screw to drop below 8 RPM's.

Preheating of the substrate fabric may be utilized as an added means of obtaining maximum adhesion of the co-extruded coating to the polypropylene fabric. The preheating can be done with open flame, cal-rod heating banks, or preferably by passing the substrate over metal heating drums that can be controlled by internal electrical or pressure stream systems to temperatures approaching 350° F.

With the extruder moved away from the threaded substrate coating line, the extrusion conditions are lined out and the die lip adjustments made to give a uniform melt at the desired output rate, die-lip opening and melt temperature (370° F.-400° F.).

With chill roll temperatures (90° F.-100° F.) and the substrate moving at a minimum speed, the extrusion line is moved into place and the coating line is quickly brought up to the predetermined line speed to deposit the required coating weight.

Adjustments in preheat control, die-to-roll distance, and roll pressure can be made to modify substrate adhesion. Coating weight is usually controlled by adjusting line speed.

The PSA preferably has a white die additive compounded into the material, which is very helpful. The white additive allows for easier visual monitoring of the die curtain. The curtain should flow completely out to the end of both deckle bars and should be consistently white throughout the entire curtain. Run minimum air gap and run the dies as close to the laminating nip as possible, as this will help stabilize the poly curtain and also avoid pulling the curtain out of the die. Run the poly curtain right at the very edge of the material. Do not run your curtain over the edges of the material, the PSA is very sticky and aggressive and will result in a laminated roll wrap.

The poly curtain must be run directly and straight into the laminating nip at 90° F. This will keep the curtain from dragging on the die lips, thus eliminating die whiskers which create voids and gels common in these resins.

Again, insure that the water cooling to the feed throat is turned off, this will help avoid a bridge at the feed zones.

1. A water impermeable, water vapor permeable, multi-layer composite sheet comprising:
a polypropylene layer;
a polyurethane layer; and
a thermoplastic pressure sensitive adhesive layer disposed between said polypropylene layer and said polyurethane layer.

2. The sheet of claim 1, further comprising a scrim reinforcing layer disposed between said polypropylene layer and said polyurethane layer.

3. The sheet of claim 1, wherein said sheet has a moisture vapor transmission value of greater than 35 grams/sq. meter/24 hrs.

4. The sheet of claim 3, wherein said sheet has a moisture vapor transmission value of between approximately 105 to 210 grams/sq. meter/24 hrs.

5. The sheet of claim 2, wherein said sheet has a moisture vapor transmission value of at least approximately 35 grams/sq. meter/24 hrs.

6. The sheet of claim 5, wherein said sheet has a moisture vapor transmission value of between approximately 105 to 210 grams/sq. meter/24 hrs.

7. The sheet of claim 1, wherein said polypropylene layer is woven.

8. The sheet of claim 1, wherein said polypropylene layer is non-woven.

9. The sheet of claim 1, wherein said pressure sensitive adhesive layer is composed of a styrene block copolymer.

10. The sheet of claim 1, wherein said polyurethane layer is approximately from about 0.00055 to 0.00060 inches thick and said pressure sensitive adhesive layer is approximately from about 0.00015 to 0.00035 inches thick.

11. The sheet of claim 1, wherein said polyurethane layer and said pressure sensitive adhesive layer are co-extruded layers.

12. The sheet of claim 1, further comprising a layer composed of a material having a high coefficient of friction disposed on said polypropylene layer opposite from said pressure sensitive adhesive layer.

13. The sheet of claim 12, wherein said layer composed of a material having a high coefficient of friction is composed of ethylenemethylacrylate.

14. The sheet of claim 12, further comprising a scrim reinforcing layer disposed between said polypropylene layer and said polyurethane layer.

15. The sheet of claim 12, wherein said sheet has a moisture vapor transmission value of greater than 5 perms.

16. The sheet of claim 15, wherein said sheet has a moisture vapor transmission value of between approximately 105 to 210 grams/sq. meter/24 hrs.

17. The sheet of claim 14, wherein said sheet has a moisture vapor transmission value of at least approximately 35 grams/sq. meter/24 hrs.

18. The sheet of claim 17, wherein said sheet has a moisture vapor transmission value of between approximately 105 to 210 grams/sq. meter/24 hrs.

19. The sheet of claim 12, wherein said polypropylene layer is woven.

20. The sheet of claim 12, wherein said polypropylene layer is non-woven.

21. The sheet of claim 12, wherein said pressure sensitive adhesive layer is composed of a styrene block copolymer.

22. The sheet of claim 12, wherein said polyurethane layer is approximately from about 0.00055 to 0.00060 inches thick and said pressure sensitive adhesive layer is approximately from about 0.00015 to 0.00035 inches thick.

23. The sheet of claim 12, wherein said polyurethane layer and said pressure sensitive adhesive layer are co-extruded layers.

24. The sheet of claim 1, wherein said sheet remains water permeable when contacted by surfactants.

25. The sheet of claim 12, wherein said sheet remains water permeable when contacted by surfactants.

26. A method of forming a water impermeable, water vapor permeable, multi-layer sheet comprising the steps of:
providing a polypropylene sheet;
co-extruding a polyurethane and thermoplastic pressure sensitive adhesive layer onto said polypropylene sheet.

27. The method of claim 26, further comprising the step of providing a reinforcing scrim layer disposed on said polypropylene sheet and co-extruding said polyurethane and thermoplastic pressure sensitive adhesive layer onto said scrim layer and said polypropylene sheet.

28. The method of claim 27, further comprising the step of extruding a material having a high coefficient of friction onto said polypropylene sheet on the side opposite to said polyurethane and thermoplastic pressure sensitive adhesive layer.

29. The method of claim 26, wherein said polyurethane layer is co-extruded at a thickness of approximately from about 0.00055 to 0.00060 inches and said pressure sensitive adhesive layer is co-extruded at a thickness of approximately from about 0.00015 to 0.00035 inches.

30. A method of forming a water impermeable, water vapor permeable, multi-layer sheet comprising the steps of:
providing a reinforcing scrim layer;
extruding a polyurethane layer onto said scrim layer;
providing a polypropylene sheet;
extruding a thermoplastic pressure sensitive adhesive layer between said composite polyurethane and scrim layer and said polypropylene sheet.

31. The method of claim 30, further comprising the step of extruding a material having a high coefficient of friction onto said polypropylene sheet on the side opposite to said polyurethane and thermoplastic pressure sensitive adhesive layer.

32. The method of claim 30, wherein said polyurethane layer is extruded at a thickness of approximately from about 0.00055 to 0.00060 inches and said pressure sensitive adhesive layer is extruded at a thickness of approximately from about 0.00015 to 0.00035 inches.

33. A method of forming a water impermeable, water vapor permeable, multi-layer sheet comprising the steps of:
providing a polyurethane sheet;
providing a reinforcing scrim layer disposed on a polypropylene sheet;
extruding a thermoplastic pressure sensitive adhesive layer between said polyurethane sheet and said composite scrim layer and polypropylene sheet.

34. The method of claim 33, further comprising the step of extruding a material having a high coefficient of friction onto said polypropylene sheet on the side opposite to said polyurethane and thermoplastic pressure sensitive adhesive layer.

35. The method of claim 33, wherein said polyurethane sheet is provided at a thickness of approximately from about 0.00055 to 0.00060 inches and said pressure sensitive adhesive layer is extruded at a thickness of approximately from about 0.00015 to 0.00035 inches.
